(19) United States
(12) Patent Application Publication
(10) Pub. No.: US 2018/0311452 A1
(43) Pub. Date: Nov. 1, 2018

(54) ASSISTED VENTILATION SYSTEMS AND METHODS

(71) Applicant: Stryker Corporation, Kalamazoo, MI (US)

(72) Inventor: Robert G. Walker, Seattle, WA (US)

(21) Appl. No.: 15/967,404

(22) Filed: Apr. 30, 2018

Related U.S. Application Data
(60) Provisional application No. 62/492,008, filed on Apr. 28, 2017.

(51) Int. Cl. A61M 16/00; A61M 16/06

(52) U.S. Cl. CPC A61M 16/0003; A61M 16/022; A61M 16/0078; A61M 2230/432; A61M 16/06

(57) ABSTRACT

Ventilation systems and/or methods are disclosed in which a cadence for the administration of ventilations is provided, selected and/or input. A notification is output at the completion of an interval based on the cadence. A subsequent interval can be started upon completion of a prior interval or can be started based on an indication that a ventilation has been administered. The elapsed time, from the beginning of the interval, and/or expiry of the interval, to the indication that a ventilation has been administered, can be tracked, monitored and/or displayed. Such indication can be provided by a user and/or based on sensor data indicative of a ventilation administration, and the indication can also indicate that an expected ventilation was not administered.

### CROSS-REFERENCE TO RELATED APPLICATIONS

This patent application claims the benefit of and priority to U.S. Provisional Patent Application Ser. No. 62/492,008 filed on Apr. 28, 2017 entitled “Method for Providing Rate Guidance for Manual Positive Pressure Ventilation,” the contents of which are hereby incorporated by reference in their entirety.

### BACKGROUND

The administration of assisted ventilations to a patient can be a crucial part of a patient's treatment, especially during an emergency medical event, such as respiratory failure, respiratory arrest, or cardiac arrest. Assisted ventilation of the patient can be performed using mechanical ventilation, but most commonly during emergency medical events, assisted ventilation is performed via manual ventilation by a user, such as a rescuer. The ventilations are typically administered spaced apart by an interval and the interval is usually timed by the rescuer. Timing of the ventilation interval can be as simple as counting by the rescuer and/or it can be assisted by a device, such as a visual or audio metronome, that provides a transient, repeated and regularly timed output. However, both the self-counting by the rescuer and/or the repeated, regular time output are not always optimal, especially during emergency situations where stress is high and many activities can be occurring simultaneously. When rescuers are distracted or have to perform other tasks, they may lose track of their self-counting and assisted ventilations may not be administered in the proper, or desired, cadence, rhythm, or rate. Similarly, the rescuer may be distracted and can miss one or more of the transient, repeated, regularly timed outputs and thus not administer assisted ventilations according to the desired cadence or at the desired rate. Whether ventilation timing is guided by a transient, repetitive indicator such as a visual or audio metronome, or by self-counting, momentary distractions can cause a rescuer to lose their place within the interval between ventilations, creating uncertainty about whether the time of the next intended breath delivery is in the future, or has already passed. Additionally, during stressful events, perception of elapsing time can become distorted, and the rescuer may be unaware that the rate at which they are actually delivering ventilations is deviating significantly from the rate at which they are intending to deliver ventilations. During cardiac arrest, and other emergency medical events, the adherence to a specific appropriate ventilation rate, or cadence, can be vital for achieving and maintaining ventilatory homeostasis, as well as minimizing negative impacts on other aspects of the patient's physiology.

There exists a need for systems and/or methods that improve the administration of ventilation to a patient according to a desired and controlled cadence.

### BRIEF DESCRIPTION OF THE DRAWINGS

FIG. 1 is an example ventilation system.

FIG. 2 is another example ventilation system.

FIG. 3 is yet another example ventilation system.

FIG. 4 is an example cadence device.

FIG. 5 is a block diagram of an example cadence device.

FIG. 6 is an example ventilation method.

FIG. 7 is another example ventilation method.

### DETAILED DESCRIPTION

Ventilation systems and methods are described herein. The systems and/or methods can assist with the timing, or cadence, of administering assisted ventilations to patients. The cadence can be a default cadence, selected from one or more predetermined cadences and/or the cadence can be provided/modified by a user, device and/or system. The cadence can include an interval that spaces apart the administration of ventilations. A cadence device can include a timer to time the interval and the timer can be reset, at the completion of the interval, to time a subsequent interval. Alternatively, or additionally, the reset of the timer can be based on an input indicating that a ventilation has been administered. The indication of a ventilation having been administered can be based on a user input and/or sensor data. The sensor data can be from sensors monitoring a device/system used for the administration of the ventilations and/or from sensors monitoring the physiologic state of the patient. For example, airway and/or CO₂ sensors can provide sensor data indicative of a ventilation being administered to the patient. Further, the cadence can be modified during a treatment session of the patient, such as to alter a length/duration of the interval. During the interval and/or at completion of the interval, the cadence device can provide a notification and/or output to indicate that a ventilation should be administered based on the cadence. Additionally, the cadence device can track, monitor and/or continuously display the elapsing time from the beginning, and/or expiry, of the interval to the indication of the administration of a ventilation. This monitoring/display can provide a continuous indication to a user and/or rescuer of where they are in the intended ventilatory cycle, and also whether an expected ventilation has been administered or not. The cadence device can be a singular or distributed device and/or the functions/features of the cadence device can be integrated with one or more other devices/systems.

Assisted ventilations are provided to a patient by forcing air into the lungs of the patient to assist and/or provide respiration for the patient. Assisted ventilations can be delivered by a variety of methods, such as by mouth-to-mouth resuscitation and/or by the use of a device/system. An example device/system can include a self-inflating resuscitation, or ventilation, bag. This bag can be connected to a ventilation mask which is placed over the mouth and nose of the patient, or can be connected to an endotracheal tube or extraglottic airway device that is inserted into and/or through the patient's upper airway. A user can compress the ventilation bag to cause air from the ventilation bag to be passed directly into the endotracheal tube or extraglottic airway device and into the patient's lungs, or alternately into the ventilation mask, through the patient's mouth and/or nose, and into the patient's lungs. Ventilation of a patient can be administered during many different emergency care conditions to aid in the treatment of the patient. Ventilation of the patient can be by assisted ventilation via one or more methods, devices and/or systems, such as those discussed herein.

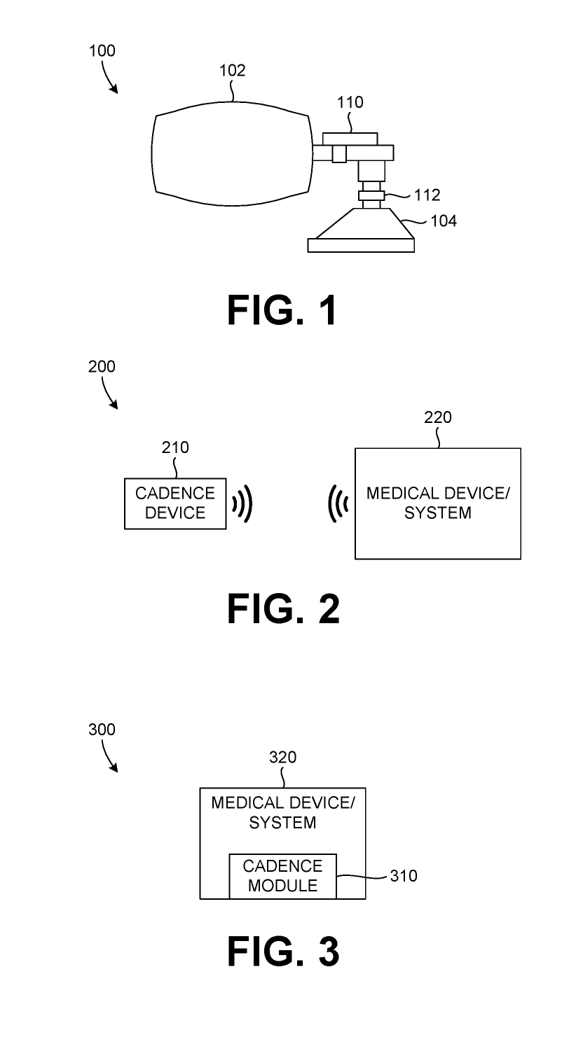

FIG. 1 is an example ventilation system 100 that includes a ventilation bag 102, a ventilation mask 104 and a cadence device 110. The ventilation mask 104 may alternately be an endotracheal tube or extraglottic airway device, such as described above. The cadence device 110 can attach to the ventilation bag 102 and/or mask 104 to assist a user with administration of ventilations by the ventilation bag 102 and mask 104. The cadence device 110 can alternately be a component of an accessory, such as an airway sensor or airway adjunct 112, which is also attached to the bag 102 and/or mask 104. The airway sensor or adjunct 112 may be, for example, an airway CO₂ sensor, and airway pressure or flow sensor, a positive end-expiratory pressure (PEEP) valve, or an impedance threshold device (ITD). Such sensors and adjuncts can attach to the ventilation bag 102, and/or the ventilation mask 104 in various ways. To assist with timing, or spacing, of the administration of ventilations, the cadence device 110 can output a notification to the user to administer a ventilation via the ventilation bag 102 and mask 104 based on a predetermined schedule, on one or more inputs and/or a combination thereof. To additionally assist with timing, or spacing, of the administration of ventilations, the cadence device 110 can continuously display or otherwise indicate the elapsing time during the interval between scheduled ventilations.

The cadence timing of the cadence device 110 can be input, or selected, by a user and/or can be a default, predetermined timing. Once started, the timer of the cadence device 110 can countdown, or count up, a time interval based on the cadence timing. Upon expiry of the timer, the cadence device can output the notification to administer a ventilation. The timer can then reset, or begin, to countdown the interval to the next ventilation administration according the to the cadence timing. The cadence device 110 can include multiple cadence timings from which the user can select and/or can include a default cadence timing that is used unless another cadence timing is selected or input. The user can also input their own cadence timing and/or modify an existing cadence timing. Additionally, the cadence timing can be modified and/or changed during a treatment session.

The cadence device 110 can include one or more inputs to allow the user to interact with the cadence device 110, such as to activate the cadence device, select a cadence timing and/or interact with one or more functions and/or features of the cadence device 110. Example inputs can include one or more buttons, a speech input, such as a microphone, an electronic communication input, such as to another device, and/or other input(s) to allow a user to interact with the cadence device 110 and/or one or more functions/features thereof. Such inputs can be on the cadence device, and/or can be on any other communicatively coupled device.

The cadence device 110 can also include an input to reset the timer. For example, the cadence timing can include an interval to space ventilations, however, upon expiry of the timer, the subsequent countdown, or count up, interval may not commence until an input is received to cause the timer to start the subsequent countdown interval. In this manner, an input is required to reset the timer of the cadence device. In the absence of such input, the timer may continue to count elapsed time until such input is provided. The input to reset the timer can be provided by a user, such as upon administration of a ventilation, by a sensor signal, such as a signal indicating the administration of a ventilation, and/or by one or more other sources, such as another device and/or system. Signals indicating administration of a ventilation could include, for example, airway CO₂, airway pressure, airway flow, or a signal derived from squeezing or deformation of the ventilation bag. For signals indicating administration of a ventilation, the trigger to reset the timer can be the sensing of the inspiratory phase of the ventilation cycle, or alternately can be the sensing of the expiratory phase of the ventilation cycle. Additionally, the cadence device can track and/or output the elapsed time between the ventilations and/or the elapsed time between the expiry of the timer and the input. Also, the cadence device 110 can also track the total time of the treatment session from the moment the cadence device 110 was initiated to begin providing cadence timing to the termination of the treatment session.

The cadence device 110 can also track the number of ventilations administered, the timing of each ventilation, and/or the interval between each ventilation. The number of ventilations administered, timing of ventilations, and/or interval between ventilations can be displayed on a display of the cadence device 110, transmitted to a communicatively coupled device, or otherwise output to a user. The cadence device 110 can additionally track the timing guidance settings and any setting changes applied during use of the device. The cadence device 110, and/or a communicatively coupled device, can further compare the actual timing, rate, and/or regularity of delivered ventilations to the timing guidance provided to the rescuer, in order to measure and/or report the accuracy and precision of the delivered ventilations.

The various timing information of the cadence device 110 can be stored by the cadence device 110 and/or transmitted to an external device and/or system. This can allow the timing information of the treatment session to be stored and/or reviewed.

The ventilation administration notification can include a notification to administer the ventilation upon expiry of the timer and/or can include a continuously displayed progressive indicator indicative of the cadence timing and the elapsed time within a ventilation cycle. To provide the ventilation administration notification, and/or cadence timing, to a user, the cadence device 110 can include one or more outputs, such as a visual, haptic, and/or an audible output. Example visual outputs of the cadence device 110 can include a display and/or lights to communicate the notification and/or the cadence timing. Example audible outputs of the cadence device 110 can include a speaker and/or an annunciator to communicate the notification and/or the cadence timing.

The cadence device 110 can be a selectively removable device that can be attached to the ventilation bag 102 and/or mask 104 prior to and/or during administration of treatment to a patient. Once initiated, the cadence device 110 can assist a user, such as a rescuer, with administration of ventilations to the patient by tracking the administration of ventilations, tracking and displaying the elapsed time between the intended timing of ventilations, and/or providing notifications to administer ventilations based on the cadence timing of the cadence device 110.

FIG. 2 is another example ventilation system 200 that include a cadence device 210 that can communicate with a medical device and/or system 220. In the example system 200, the cadence device 210 can be a standalone device that provides ventilation assistance, such as that discussed above. The cadence device 210 can communicate with a medical device 220, via a wired and/or a wireless connection, to receive various information, such as patient physiological information, that can be used to track the administration of ventilations and/or to assist with the cadence timing. For example, the medical device 220 can communicate physiological information from which the cadence device 210 can determine administration of a ventilation, and/or the medical device 220 can communicate an indication of a ventilation administration to the cadence device 210. The determination of the administration of a ventilation can be an input to the cadence device 210 to cause a timer of the cadence device 210 to reset and begin timing another interval to a further ventilation administration. The cadence device 210 can also provide the ventilation notification to the medical device 220, which can then output the notification to the user. In an example, one or both of the cadence device 210 and the medical device 220 can provide the notification.

As a standalone device, the cadence device 210, and/or its functionality/features, or portions thereof, can be integrated with another device. For example, the cadence device 210 can be an application on a mobile device, such as a cell phone, smartwatch, or tablet. In this manner, the cadence device 210 can be integrated with one or more other devices and/or systems, which can minimize the number of devices a rescuer can be required to monitor and/or track. In an example, the integrated cadence device 210 can communicate with one or more other devices and/or systems via a wired and/or a wireless communication to exchange information, such as cadence timing, detection of delivered ventilations, physiologic state, and/or other data.

FIG. 3 is another example ventilation system 300 that includes a medical device/system 320 having an integrated cadence module 310. The cadence module 310 can provide the same, or similar, functionality/features as the cadence device(s) discussed above. In the system 300, the functionality and/or features of a cadence device are integrated with the medical device 320, rather than having a separate cadence device. The cadence module 310 can receive various information from the medical device 320, such as patient physiological data and/or detection of delivered ventilations, to assist with the cadence timing and/or monitoring, such as tracking the administration ventilations and/or elapsed timing between ventilations. Additionally, the cadence module 310 can provide the ventilation notification and/or other ventilation data via one or more outputs of the medical device 320, such as a screen and/or speaker.

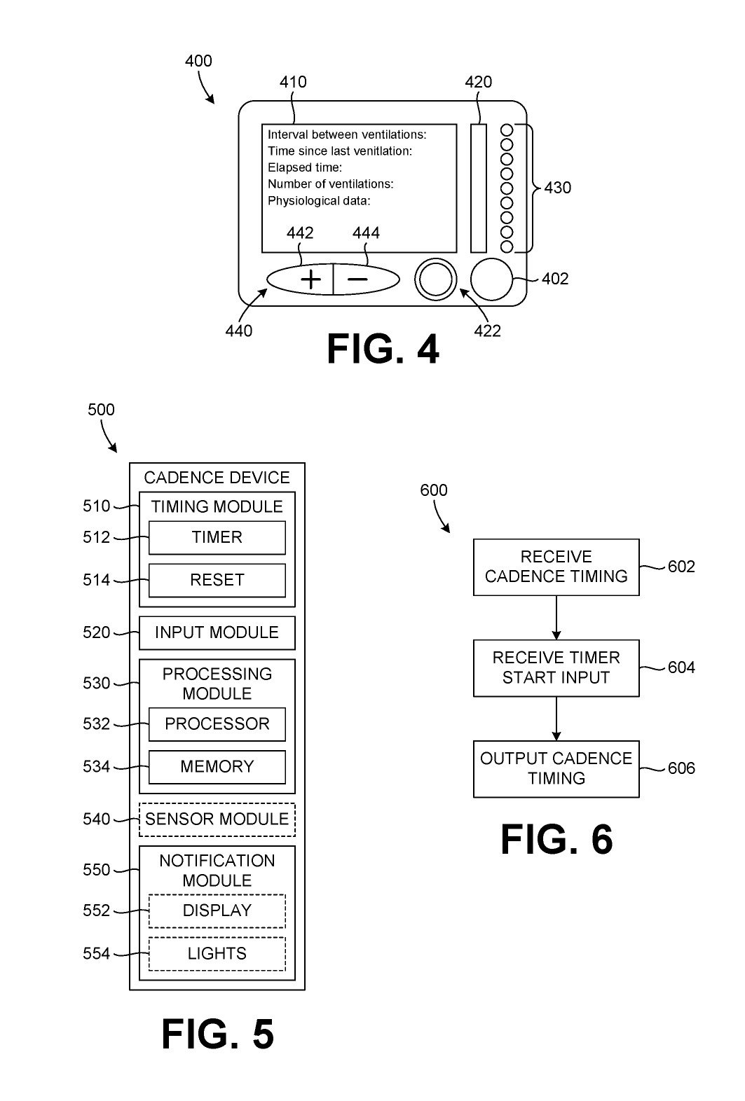

FIG. 4 is an example cadence device 400 that includes one or more outputs, such as a display 410, a bar indicator 420, a circular indicator 422, and/or lights 430, and one or more inputs, such as a power or initiation button 402 and an adjustment input 440. Further and/or alternative input(s) and/or output(s) can be included in/on the cadence device 400.

The display 410 can be a graphical display that can provide various information to a user, such as a rescuer. For example, the display can provide a countdown to the next ventilation, an indication of time since the last ventilation and/or expiry of the timer, the number of ventilations administered, the cumulative duration of ventilation assistance, physiologic data, such as EtCO₂, from other connected or communicatively coupled devices, and/or other information. Additionally, the display 410 can provide an interface to allow a user to select and/or alter one or more functions/features of the cadence device 400, such as a predetermined cadence timing. To interact with the display 410, and/or the information displayed thereon, the display 410 can include touchscreen capability to allow the user to interact with the display 410 and/or the user can interact with one or more other inputs of the cadence device, such as the adjustment input 440, to interact with the display 410. Adjustments may alternately be input on a communicatively coupled device or module.

The bar indicator 420 can provide a progressive indication of the cadence timing, such as by illuminating, or darkening, portions of the bar indicator 420 relative to the cadence timing. For example, as the timer progresses, a larger portion of the bar indicator 420 can illuminate, or darken, until the bar indicator 420 is completely illuminated, or darkened, upon expiry of the timer. Additionally, the bar indicator 420 can include color to assist with providing ventilation notification. For example, the bar indicator 420 can be divided into one or more sections that can be each assigned a color. As the bar indicator 420 progressively illuminates, or darkens, the differently colored sections are illuminated relative to the cadence timing. In another example, the bar indicator 420 can change color based on an elapsed time from expiry of the timer. The bar indicator 420 can progressively fill until the indicator 420 is fully illuminated upon expiry of the timer. The timer can require an input prior to resetting, and the bar indicator 420 can progressively change color based on the elapsed time from the expiry of the timer. In an embodiment, the bar indicator 420 can fill in a green color upon expiry of the timer, after a first elapsed time interval the bar indicator 420 can change from green to yellow and after a second elapsed time interval the bar indicator 420 can change from yellow to red, to indicate the elapsed time since the expiry of the timer without receiving an input from a user to reset the timer. In addition to changing color, the bar indicator 420 can also flash to assist with notification of the cadence timing and/or the elapsed time since expiry of the timer.

A circular indicator 422 can also be included, such as in addition to or in place of the bar indicator 420. The circular indicator can include an outer ring that can be filled, or illuminated, as the timer countdowns to provide an indication of a remaining amount of time until expiry of the timer. Alternatively, the outer ring can start filled, or illuminated, and proceed to an unfilled, or non-illuminated, state relative to the timer. Additionally, the outer ring can include multiple illumination colors and/or can change colors of illumination relative to the remaining time until expiry of the timer. Further, the center portion of the circular indicator 422 can also illuminate. This center portion can illuminate, dim and/or change color relative to the remaining time until expiry of the timer. In another embodiment, the center portion can illuminate upon expiry of the timer to indicate that a ventilation should be administered according to the ventilation timing. As time continues past the expiry of the timer, the center portion can change color, and/or display text, to indicate the elapsed time past the expiry of the timer and/or the elapsed time since the last ventilation.

The lights 430 can operate similarly to the bar indicator 420, with a number of the lights 430 progressively illuminating relative to the cadence timing. Additionally, the lights 430 can be divided into sections that can each be assigned a color of illumination. Further, the lights 430 can be flashed to assist with notification of the cadence timing and/or the elapsed time since expiry of the timer.

The power/initiation button 402 can be actuated by a user to activate the cadence device 400 and/or to initiate the cadence timing, such as based on a default cadence timing, a selected cadence timing and/or a user input/modified cadence timing. The button 402 can be a multi-function button that can be used to interact with one or more functions/features of the cadence device 400, such as based on a length of actuation. For example, a long actuation of the button 402 by a user can cause the cadence device 400 to power on or off, and a short actuation can cause the cadence device 400 to initiate cadence timing. Additionally, actuation of the button 402 can also allow a user to access one or more functions/features of the cadence device 400 that can be modified by the user.

The adjustment input 440 can allow a user to provide an input to alter one or more functions/features of the cadence device 400, such as the cadence timing. The adjustment input 440 can include a first button 442 and a second button 444 that can be actuated by a user. In an example, to alter a cadence timing, a user can actuate the first button 442 to extend a cadence timing interval, the time interval between ventilation administrations, and/or actuate the second button 444 to reduce the cadence timing interval. Additionally, or alternatively, the adjustment input 440 can be actuated to modify other characteristics and/or properties of the cadence timing, such as selecting a cadence timing from one or more predetermined cadence timings displayed on the display 410 and/or indicated by the bar indicator 420 and/or lights 430. In an alternate embodiment, adjustments can be made on a communicatively coupled device, and transmitted to the cadence device 400.

In an example embodiment in which the timer of the cadence device 400 is reset by an input, one or more of the buttons 442, 444 of the adjustment input 440 and/or the button 402 can be actuated by a user to provide the input to reset the timer. Additionally, or alternatively, the cadence device 400 can receive the input from an external device and/or system, such by an indication of a ventilation administration based on one or more sensor signals from one or more sensors monitoring a patient.

FIG. 5 is an example cadence device 500 that includes a timing module 510, an input module 520, a processing module 530, an optional sensor module 540, and a notification module 550. The cadence device 500 provides, or outputs, cadence timing to assist with the administration of ventilations. A user, device and/or system can administer ventilations to a patient based on the cadence timing provided by the cadence device 500. The cadence timing can be a user, device and/or system provided, or modified, timing schedule and/or a predetermined timing schedule. The timing schedule, or cadence timing, is a desired time interval that separates administration of ventilations. For example, a predetermined cadence timing could be the administration of a ventilation at six-second intervals. The cadence device 500 counts down, or up, the six-second interval and provides a notification to administer the assisted ventilation upon expiry of the six-second interval. In an alternate embodiment, when ventilation is being provided to a patient in cardiac arrest and receiving CPR, the predetermined and/or user configured cadence timing can be based upon a specific number of elapsed chest compressions, rather than a specific number of elapsed seconds. Detection and counting of chest compressions may be accomplished via sensors connected to the cadence device and/or sensors connected to a separate communicatively coupled device. Additionally, the cadence device 500 can track and/or display the elapsed time since the current time interval commenced, which allows a user, device and/or system to determine a total elapsed time since the previously administered ventilation. Further, the cadence device 500 can track information such as the number of ventilations administered during a treatment session, the duration over which ventilations were provided, and the rate(s) at which ventilations were provided.

The timing module 510 includes a timer 512 and a reset 514. The timer 512 times the cadence timing, which can include timing the interval spacing ventilation administrations, the elapsed time since the last ventilation administration and/or the elapsed time since expiry of the interval. The elapsed time(s) between ventilation administrations can also be recorded and/or stored in a memory 534 of the cadence device 500. The reset 514 can reset the timer 512 to begin timing another interval spacing the ventilation administrations. The reset 514 can be automatic, such as upon expiry of the timer 512 and/or in response to an input, such as received at, or by, the input module 520. In an example, when the timer 512 completes timing an interval of the cadence timing, the reset 514 causes the timer 512 to time a further interval of the cadence timing. In a further example, the reset 514 can require an input before resetting the timer 512, to allow the timer 512 to time a further interval of the cadence timing. The input can be provided by a user, device and/or system and/or can be based on a determination that a ventilation has been administered. A user can provide the input, such as an indication that a ventilation has been administered by the user, to the reset 514 to cause the timer 512 to be reset. Alternatively, or additionally, the determination that a ventilation has been administered can be based on sensed physiological parameter data of the patient, such as by an airway and/or CO₂ sensor, or can be based on data from a sensor on the ventilation bag, such as a sensor that detects when the bag is squeezed or deformed. The cadence device 500 can evaluate the physiological data to determine the administration of a ventilation and/or can receive an indication of the determination of the administration of a ventilation from another device and/or system. In response to determining a ventilation has been administered, based on the physiological parameter data, an input can be provided to the reset 514 to reset the timer 512.

The input module 520 can allow information and/or data to be provided to the cadence device 500 by a user, device and/or system. The input module 520 can include an interface that can be interacted with by a user to provide input to the cadence device 500, such as to initiate a cadence timing, to select a cadence timing, to modify a cadence timing and/or to provide other input to one or more functions/features of the cadence device 500. Additionally, or alternatively, the input module 520 can include a communications capability to allow the cadence device 500 to receive input from, and/or to communicate with, another device and/or system.

The processing module 530 can include a processor 532 and memory 534. The processor 532 can control one or more functions/features of the cadence device 500 and/or process data received and/or sent by the cadence device 500. The memory 534 can store instructions for execution by the processor 532 and/or can store various data of the cadence device 500. In an example, the memory 534 can store one or more predetermined cadence timings. A user can select, and/or modify, one of the predetermined cadence timings of the memory 534, such as by the input module 520. After the cadence timing has been selected, and/or modified, the processor 532 can provide the cadence timing from the memory 534 to the timing module 510 to begin timing according to the provided cadence timing.

The cadence timing can be constant, with the interval between ventilations fixed to a specific length of time. Alternatively, the cadence timing can be variable, with the interval between ventilations being varying lengths of time. In the variable embodiment, the varying lengths of time can be a repeated pattern, such as a first interval and a second interval that repeat, or can be a variable pattern. In the variable pattern embodiment, the interval can be based on various input to the cadence device 500, such as based on patient physiological parameter data and/or user, or other, provided input.

A user, device and/or system can also, optionally, modify the cadence timing during a treatment session. For example, a user can initially select a first cadence timing at the beginning of a treatment session, then can select a second cadence timing during the treatment session, such as in response to a physiologic state of a patient being treated.

The sensor module 540 can include one or more sensors and/or can receive data from one or more sensors external the cadence device 500. In an example, the sensor module 540 can include one or more sensors that can monitor the cadence device 500 and/or a patient to which treatment is being administered. In an embodiment, the reset 514 can require an input to reset the timer 512 and the input can be an indication of the administration of a ventilation. The cadence device 500 can be attached, and/or connected, to a ventilation bag for administration of ventilations and a sensor(s) of the sensor module 540 can monitor the ventilations administered by the ventilation bag. When administration of a ventilation is determined, such as from the sensor data, the sensor module 540 can provide an input to the reset 514 and/or the processing module 530 to indicate the administration of a ventilation which can be used to reset the timer 512. The sensor module 540 can also receive data from sensors monitoring a physiologic state of the patient. The sensor data collected by monitoring the physiologic state of the patient can also be used to provide, or generate, the input to the reset 514. The reset can occur automatically or on demand. For example, the timer can automatically reset to begin another cycle of the same or a different timing to indicate the administration of ventilations or can output a prompt to a user to reset the timing. In the automatic examples, the timer can automatically reset to the previously performed timing interval or to a default or other timing, as needed, or as predetermined.

For example, airway pressure and/or capnography sensor data can be used to determine the administration of a ventilation to the patient. This determination can be provided, and/or used to provide, the input to the reset 514. Additionally, the cadence timing of the cadence device 500 can be modified and/or set based on the collected physiological parameter data of the patient, such as automatically by the cadence device 500, or other, device/system, and/or by a user. The physiological parameter data, and/or instructions to alter the cadence timing, can be received from an external device/system that is monitoring and/or treating the patient, such as a patient monitor or a monitor-defibrillator. For example, a first cadence timing can be preselected, or selected by the user or system at the beginning of a treatment session. Then, if EtCO₂ values, as measured for example by an attached sensor or transmitted from a separate communicatively coupled device, increase above a preconfigured threshold, the cadence timing can automatically switch to a second, shorter, interval. In a similar manner, if EtCO₂ values decrease below a preconfigured threshold, the cadence timing can automatically switch to a different, longer, interval.

The notification module 550 can include a display 552 and/or lights 554 to provide notification to a user based on the cadence timing. The display 552 can display various information, such as a number of ventilations administered, the cumulative duration over which ventilation has been provided, the cadence timing, notification of the expiry of the interval spacing ventilation administrations, time elapsed since expiry and/or beginning of the timer, physiologic data from other connected or communicatively coupled devices, and/or other information. The display 552 can also display information indicative of one or more aspects of the quality with which ventilation is being provided, or has been provided, such as the regularity of the delivered ventilations, the concordance between the ventilation timing guidance provided by the cadence device and the actual delivered ventilations, and the concordance between a preconfigured target range of EtCO₂ and actual EtCO₂ levels achieved during ventilation. The display 552 can present the various information in one or more graphical formats, which can be, optionally, configured by a user. In addition to the cadence/treatment session information, the display 552 can display various menus for the functions and/or features of the cadence device 500 to allow the user to configure the cadence device 500 and/or the cadence timing.

The lights 554 can be located about the cadence device 500 and can provide indications regarding the cadence timing. In an example, an increased number of lights can be illuminated as time elapses during the timer interval, and/or as the timer reaches expiry. Additionally, the lights 554 can be colored, and/or capable of changing color, such as to provide indications of a quality, like an importance or severity, based on the color of the illuminated lights 554. Further, the lights 554 can be illuminated in a pattern, such as repeated flashing, to provide additional indication and/or notification beyond the illumination of the lights 554.

The cadence device 500 is shown as a singular device, however, the various elements, systems, functions and/or features of the cadence device 500 can be distributed across one or more devices. Additionally, or alternatively, the various functions and/or features of the cadence device 500 can be integrated with and/or provided by another device/system, such as another medical device or a mobile computing device.

FIG. 6 is an example ventilation method 600 based on cadence timing. At 602, cadence timing can be received. The cadence timing can include an interval, pattern of intervals and/or other interval information, for spacing apart of the administration of ventilations. At 604, a timer start input can be received. The start input can cause the timer to begin timing, or counting down or up, the interval based on the cadence timing. At 606, the cadence timing can be output. The output of the cadence timing can include a notification at the expiry, or completion, of each interval. The notification can be received by a user, device and/or system to cause the administration of a ventilation in response to the notification. Additionally, the output 606 can include monitoring, tracking and/or displaying an elapsed time for each interval and/or an elapsed time since expiry/completion of the timing for each interval.

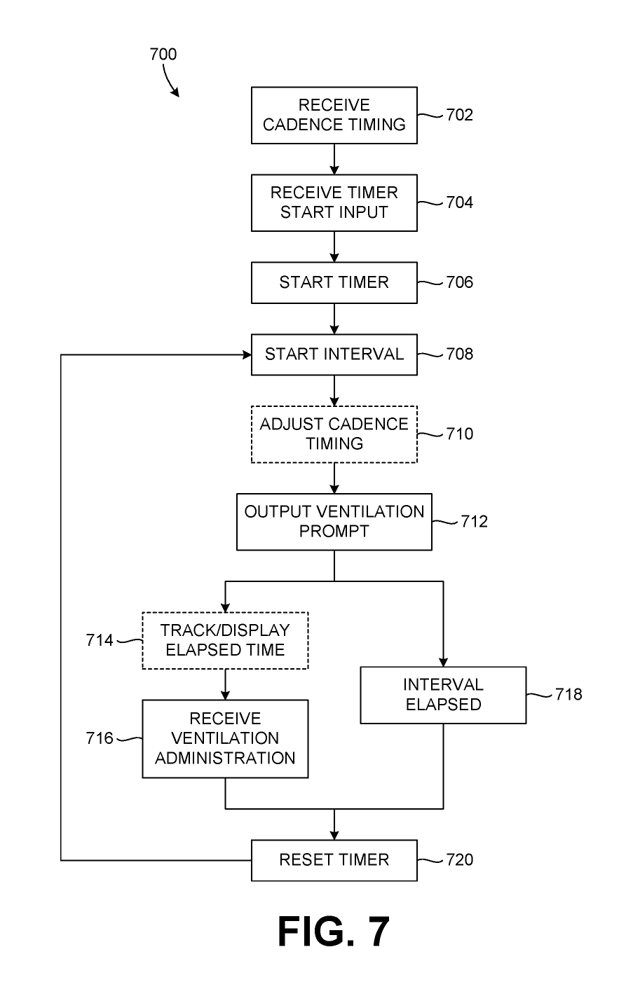

FIG. 7 is an example ventilation method 700. At 702, cadence timing can be received. The cadence timing can include a fixed and/or variable interval for spacing apart the administration of ventilations. At 704, a timer start input can be received to begin timing according to the cadence timing. At 706, a timer can be started. The timer can time each of the intervals according to the cadence timing. At 708, the interval can be started. The timer can time the duration of the interval based on the cadence timing. At 710, optionally, the cadence timing can be adjusted, such as by a user, device and/or system. The adjustment of the cadence timing can be based on the physiologic state and/or physiological data of a patient to which the ventilations are being administered. At 712, a ventilation prompt can be output. The output of the ventilation prompt is provided substantially at the end of the interval, based on the cadence timing, being timed by the timer. The notification can include a visual, audible and/or tactile notification to a user, device and/or system to administer a ventilation based on the cadence timing.

At 714, the elapsed time can be, optionally, tracked and/or displayed. The elapsed time can be from the beginning of the interval 708 or from the output of the prompt at 712 upon expiry, or completion, of the interval. The elapsed time can provide an indication to a user, device and/or system that an expected ventilation has not yet been administered and a duration since the last ventilation was administered, or since the ventilation was expected to be administered. At 716, a ventilation administration can be received. Receiving the ventilation administration can include receiving an input indicating that a ventilation was administered. This input can be provided by a user, such as a user indicating that they, or a device/system, has administered a ventilation, or can be provided based on data from one or more sensors. In an example, a sensor can monitor the device/system used to administer the ventilation and can provide sensor data indicative of the administration of a ventilation by the device/system. This sensor data can be used as, or to provide, the input to indicate that a ventilation has been administered. Additionally, or alternatively, one or more sensors can monitor the patient and can provide sensor data indicative of a ventilation received by the patient. For example, an airway and/or capnography sensor can provide sensor data indicating that a ventilation was administered to the patient. The indication and/or sensor data can be provided to be used, or to generate, the input to indicate that a ventilation has been administered.

Alternatively, or additionally, at 718, the interval can elapse, complete or expire. That is, the interval being timed can have been completed. At 720, the timer can be reset and another interval can be started at 708 to continue the ventilation treatment of the patient.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing the invention in diverse forms thereof.

1. A cadence device for use during ventilation treatment of a patient, comprising:
an input module configured to receive one or more inputs relating to the ventilation treatment of the patient;
a processing module configured to:
determine a cadence timing based at least in part on the received one or more inputs; and
generate a timing instruction that includes a timer start input, the cadence timing, and a timer interval;
a timing module having a timer and a reset, the timer configured to:
receive the timing instruction;
start the timer according to the timer start input of the timer instruction;
instruct the timer to time according to the cadence timing;
reset the timer at an end of the timer interval; and
generate one or more ventilation notifications based at least in part on one or more of the timer start input, the cadence timing, the timer interval, and the reset of the timer at the end of the timer interval; and
an notification module configured to output the one or more ventilation notifications.

2. The device of claim 1, wherein the input module is further configured to receive the one or more inputs relating to the ventilation treatment of the patient from one or more of a user administering the ventilation treatment to the patient or one or more patient physiological sensors, airway sensors, or ventilation bag deformation sensors.

3. The device of claim 2, wherein the one or more patient physiological sensors is configured to sense patient physiological, airway, or ventilation bag deformation data indicative of ventilation administered to the patient.

4. The device of claim 3, wherein the reset of the timer at the end of the timer interval is based on the sensed patient physiological, airway, or ventilation bag deformation data indicative of ventilation administered to the patient.

5. The device of claim 1, wherein the input module is further configured to receive user input adjusting a default of one or more of the timer start input, the cadence timing, or the timer interval.

6. The device of claim 5, wherein the processing module further includes a memory that is configured to store one or more default timing instructions and the processing module is further configured to determine one or both of the cadence timing and the timer interval based at least in part on the received one or more inputs and the default timing instruction.

7. The device of claim 1, wherein the cadence timing is either fixed or variable.

8. The device of claim 1, wherein the timing module is further configured to automatically reset the timer at the end of the timing interval.

9. The device of claim 8, wherein the timing instruction is a first timing instruction and the timing module is further configured to automatically reset the timer at the end of the timing interval according to the first timing instruction.

10. The device of claim 8, wherein the timing instruction is a first timing instruction and the timing module is further configured to automatically reset the timer at the end of the timing interval according to a second timing instruction in which one or more of the timer start input, the cadence timing, and the timer interval of the second timing instruction are different than the first timing instruction.

11. The device of claim 1, wherein the notification module includes an audible or visual output.

12. The device of claim 1, wherein the notification module includes a display and the ventilation notifications are indicative of quality of the ventilation treatment of the patient.

13. The device of claim 1, wherein the ventilation notifications indicate delivery of ventilations to the patient.

14. The device of claim 1, wherein the notification module includes a display and the ventilation notifications include a progressively filling indicator.

15. The device of claim 14, wherein the progressively filling indicator includes a bar, lights, or a circle.

16. The device of claim 1, wherein the ventilation notifications of the notification module include a notification of the end of the timer interval.

17. The device of claim 1, wherein the ventilation notifications of the notification module include a notification of the reset of the timer.

18. A ventilation assistance system, comprising:
one or more patient physiological sensors configured to sense patient physiological parameter data;
a cadence module configured to determine a timing instruction for administering ventilation treatment based, at least in part, on the sensed patient physiological data, the timing instruction including a cadence timing and a timer interval;
a timer configured to time output notifications according to the cadence timing for the timer interval.

19. The system of claim 17, wherein the output notifications includes a continuous, progressive indication of elapsing time within the timer interval.

20. The system of claim 18, wherein the one or more patient physiological sensors and the cadence module are electrically coupled to a medical device.

21. The system of claim 20, wherein the cadence module is wireless connected to the medical device.

22. The system of claim 20, wherein the cadence module is integrated into the medical device.

23. The system of claim 20, wherein the timer is integrated within the medical device.

24. The system of claim 20, wherein the medical device is a patient monitor-defibrillator.

25. The system of claim 18, wherein the cadence module is a cadence device attached to a ventilation bag.

26. The system of claim 18, wherein the cadence module is a cadence device that is a standalone from the patient physiological sensors.

27. The system of claim 26, wherein the timer is integrated into the cadence device.

28. The system of claim 26, further comprising a medical device electrically coupled to the patient physiological sensors, and wherein the timer is integrated into one or both of the cadence device or the medical device.
